(19) United States
(12) Patent Application Publication
(10) Pub. No.: US 2014/0073707 A1
(43) Pub. Date: Mar. 13, 2014

(54) COMPOSITIONS COMPRISING A FLUOROSURFACTANT AND A FLUORO-FREE HYDROTROPE

(71) Applicants: Thomas G. Calvarese, Wilmington, DE (US); Weiming Qiu, Wilmington, DE (US); Anilkumar Raghavanpillai, Wilmington, DE (US); Yamaira Gonzalez, (US)

(72) Inventors: Thomas G. Calvarese, Wilmington, DE (US); Weiming Qiu, Wilmington, DE (US); Anilkumar Raghavanpillai, Wilmington, DE (US); Yamaira Gonzalez, (US)

(73) Assignee: E I DU PONT DE NEMOURS AND COMPANY, Wilmington, DE (US)

(21) Appl. No.: 13/612,960

(22) Filed: Sep. 13, 2012

(51) Int. Cl. A01N 57/12; C09D 7/12; C09D 11/00; C09K 3/00; C11D 1/58; C09K 13/00; C09G 1/18; C09K 15/30; A61K 8/69; C11D 1/34; A01N 43/40; C09K 15/20

(52) U.S. Cl. CPC A01N 57/12; A01N 43/40; C09D 7/1233; C09D 11/00; C09K 3/00; C09K 15/20; C09K 13/00; C09G 1/18; C09K 15/30; A61K 8/69; C11D 1/345; C11D 1/58
USPC 514/785; 514/788; 106/503; 106/287.25; 106/287.2; 106/31.13; 516/9; 516/12; 516/203; 252/389.22; 252/79.1; 106/3; 252/391; 252/182.3; 510/469; 510/494

(57) ABSTRACT

Compositions comprising a fluorosurtactant and a fluoro-free hydrotrope are disclosed. The fluoro-free hydrotropes are cationic aromatic compounds, anionic aromatic compounds, or water soluble azo derivatives.

### FIELD OF THE INVENTION

The present invention relates to compositions comprising a fluorosurfactant and a fluoro-free hydrotrope, and their use in surfactant applications.

### BACKGROUND OF THE INVENTION

Recent concerns over the environmental fate of fluorosurfactants and the cost of fluorosurfactants are fueling efforts to develop new surfactant systems that contain less fluorine. U.S. Pat. No. 4,089,804 discloses a method of improving fluorinated surfactants by employing a fluorinated synergist, (R_{f})ₙTₘZ, wherein R_{f} is a perfluorinated aliphatic group, T is alkylene, arylene, alkylenethioalkylene, alkyleneoxyalkylene or alkyleneiminoalkylene, Z is a neutral or a polar group, n is 1 or 2, and m is 0 to 2.

There remains a need for developing other surfactant systems having reduced fluorine content with low environmental footprint and/or improved performance.

### SUMMARY OF THE INVENTION

An aspect of the present invention relates to a composition comprising a fluorosurfactant and a fluoro-free hydrotrope, wherein the weight ratio of the fluorosurfactant to the fluoro-free hydrotrope is 1:10 to 10:1

Another aspect of the present invention relates to a method of altering the surface behavior of an agent, comprising adding to the agent a composition comprising a fluorosurfactant and a fluoro-free hydrotrope, wherein the weight ratio of the fluorosurfactant to the fluoro-free hydrotrope is 1:10 to 10:1.

A further aspect of the present invention relates to a process comprising contacting an article with a composition comprising a fluorosurfactant and a fluoro-free hydrotrope, wherein the weight ratio of the fluorosurfactant to the fluoro-free hydrotrope is 1:10 to 10:1.

### DETAILED DESCRIPTION

An aspect of the present invention relates to a composition comprising a fluorosurfactant and a fluoro-free hydrotrope. As used herein, the term “fluoro-free hydrotrope” refers to a hydrotrope comprising a fluorine-free hydrophilic part and a fluorine-free hydrophobic part. The fluoro-free hydrotropes are cationic aromatic compounds, anionic aromatic compounds, or water soluble azo derivatives. They include compounds corresponding to formulae (I), (II), and (III):

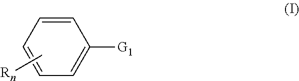

wherein:
   R=R¹ or OR¹,
      R¹=C₁-C₃ alkyl;
   n=1, 2, or 3; and
   G₁=-CO₂M, —SO₃M, —OSO₃M, —OPO₃(M₁)(M₂),
   or —PO₃(M₁)(M₂),
      M=H, Na, K, or NH₄,
      M₁=H, Na, K, or NH₄, and
      M₂=H, Na, K, or NH₄;

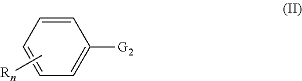

wherein:
   R=R¹ or OR¹,
      R¹=C₁-C₃ alkyl;
   n=1, 2, or 3;
   G₂=N⁺R²R³R⁴.X⁻
      R²=H or C₁-C₃ alkyl,
      R³=H or C₁-C₃ alkyl,
      R⁴=H or C₁-C₃ alkyl, and
      X=Cl, Br, or I;

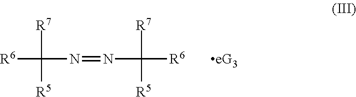

wherein:
   R⁵=C₁-C₃ alkyl;
   R⁶=C₁-C₃ alkyl; and
   R⁷=-(CH₂)ₐCO₂H,

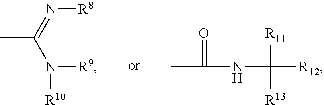

R⁸=H, —(CH₂)_{b}CO₂H, —(CH₂)_{c}OH, or C₁-C₃ alkylene,
R⁹=H, —(CH₂)_{b}CO₂H, —(CH₂)_{c}OH, or C₁-C₃ alkylene,
R¹⁰=H, —(CH₂)_{b}CO₂H, —(CH₂)_{c}OH, or C₁-C₃ alkylene,
   a=1, 2, 3, or 4,
   b=1, 2, 3, or 4,
   c=1, 2, 3, or 4,
wherein R⁸ and R⁹, or R⁸ and R¹⁰, or R⁹ and R¹⁰ may be taken together with the nitrogen atom(s) to which they are attached to form a ring containing 5 to 7 atoms,
R¹¹=H or —(CH₂)_{d}OH,
R¹²=H or —(CH₂)_{d}OH,
R¹³=H or —(CH₂)_{d}OH,
   d=1, 2, 3, or 4;
   e=0, 1, 2, 3, or 4; and
G₃=HX, H₂SO₄, or H₂O,
   X=Cl, Br, or I.

In some embodiments, the water soluble azo derivatives of formula (III) comprise 5-membered rings with one or two nitrogens. Exemplary azo derivatives of formula (III) comprising 5-membered rings are shown below as (IIIa) and (IIIb):

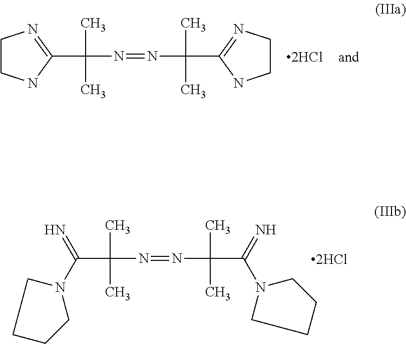 [SMILES: CC(C)(N=NC(C)(C)C1=NCCN1)C1=NCCN1 ; CC(C)(N=NC(C)(C)C(=N)N1CCCC1)C(=N)N1CCCC1]

Carboxylate derivatives in formula (I) (G₁=-CO₂M) include p-methylbenzoic acid and p-methoxybenzoic acid.

Sulfonate derivatives in formula (I) (G₁=-SO₃M) include p-toluenesulfonic acid. Suitable sulfonate derivatives can also be synthesized by sulfonation of alkylbenzenes or alkoxybenzenes, followed by treating the reaction mixture with a hydroxide solution, as per the procedure described in Hajipour et al., Tetrahedron Lett., 2004, 6607.

Sulfate derivatives in formula (I) (G₁=-OSO₃M) include 4-methoxyphenyl sulfate. Suitable sulfate derivatives can be synthesized by reacting the corresponding phenol derivatives with sulfur trioxide-pyridine complex as per the procedure described in Denehy et al., Chem. Comm., 2006, 314.

Phosphate derivatives in formula (I) (G₁=-OPO₃(M₁)(M₂)) include 4-methylphenyl phosphate. Suitable phosphate derivatives can be synthesized by reacting the corresponding phenol derivatives with POCl₃, to yield alkylphenyl or alkoxyphenyl dichlorophosphates, which are then treated with water to yield the phosphate derivatives as per the procedure described in Rapp, Justus Liebigs Ann. Chem., 1884, 162.

Phosphonate derivatives in formula (I) (G₁=-PO₃(M₁)(M₂)) include 4-methylphenyl phosphonate. Suitable phosphonate derivatives can be synthesized from bromoarenes and P(OEt)₃ as per the procedure described in Yuan, C.; Feng, H. Synthesis, 1990, 140.

Suitable anilinium derivatives in formula (II) include benzyltriethylammonium chloride, p-toluidine hydrochloride, and p-methoxyanilinium chloride. Suitable anilinium derivatives can also be made by the reaction of alkoxyanilines or alkylanilines and formaldehyde as per the procedure described in Tajbakhsh et al., Synthesis, 2011, 490.

Azo derivatives in formula (III) include those available from E. I. du Pont de Nemours and Company (Wilmington, Del.) and from Walko Pure Chemical Industries (Richmond, Va.). Examples of these derivatives are (IIIa) and (IIIb), described supra.

Water-soluble cationic, anionic, amphoteric, and nonionic fluorosurfactants can be used in this invention. “Cationic fluorosurfactants” denotes fluorosurfactants containing cationic groups and/or groups able to be protonated into cationic groups. In some embodiments, the cationic fluorosurfactant comprises primary, secondary, tertiary, and/or quaternary amine groups. In some embodiments, the cationic fluorosurfactant comprises a pyridinium group. “Anionic fluorosurfactants” denotes fluorosurfactants containing anionic groups and/or groups able to be deprotonated into anionic groups. In some embodiments, the anionic fluorosurfactant comprises carboxy group(s), sulfonic group(s), phosphate group(s), phosphonate group(s) or their corresponding salts. “Amphoteric fluorosurfactants” denotes fluorosurfactants containing at least one cationic group and at least one anionic group, as defined above for cationic and anionic fluorosurfactants. “Nonionic fluorosurfactants” denotes fluorosurfactants containing polyethylene glycol polymers, polypropylene glycol polymers, and copolymers thereof. In some embodiments, the nonionic fluorosurfactant comprises polyoxyethylene fluoroalkyl ethers, or polyoxyethylene fluoroalkylphenyl ethers. The three types of fluorosurfactants include, in particular, those corresponding to formulae (IV), (V), (VI):

A₁-(CH₂)ₘ—Y (IV)
wherein
   m=1, 2, or 3;
   A₁=linear or branched C₂-C₂₀ perfluoroalkyl; and
   Y=—CO₂M₃, —SO₃M₃, —SCH₂CH₂CO₂M₃, —OPO(ONH₄)₂, —SO₂—NH(CH₂)₃—N⁺(CH₃)₃.I⁻, —SCH₂CH₂N⁺(CH₃)₃.CH₃—SO₄⁻, —(OCH₂CH₂)ₖ—OH,

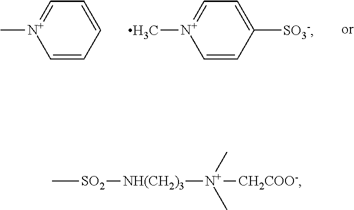

wherein
M₃=H, alkali metal, or NH₄⁺; and
k=an integer ranging from 1 to 40;

A₂-R¹⁴ (V)
wherein:
   A₂=linear or branched C₄-C₁₆ perfluoroalkyl; and
   R¹⁴=—SO₃⁻.N⁺(R¹⁵)₄, —CO₂N⁺(R¹⁵)₄, —SO₂N(R¹⁵)CH₂CO₂⁻.M₄⁺, or —SO₂—NH(CH₂)ₚ—N⁺(R²)₃.I⁻,
      wherein:
         R¹⁵=H or C₁-C₄ alkyl,
         M₄=H or alkali metal, and
         p=1, 2, 3, or 4;

(A₃C₂H₄O)ₓP(O)(R¹⁶)_{y}(R¹⁷)_{3-x-y} (VI)
wherein
   A₃=linear or branched C₃-C₈ perfluoroalkyl;
   x=1 or 2;
   y=1 or 2;
provided that:
   x+y<=3
   R¹⁶=ONH₄ or OH; and
   R¹⁷=OC₂H₄OH;

Suitable fluorinated surfactants include those available from E. I. du Pont de Nemours and Company (Wilmington, Del.) under the trade names Zonyl®, Capstone®, and Forafac® and from 3M Company (Minneapolis, Minn.) under the trade name Fluorad®.

Some examples of suitable fluorosurfactants according to formulae (IV), (V), and (VI) are shown in Table 1.

TABLE 1

| | |
|---|---|
| A₁—CH₂CH₂—SO₂—NH(CH₂)₃—N⁺(CH₃)₃•I⁻ | Formula (IV) |
| 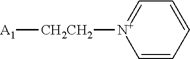 | |
| 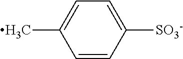 | |
| 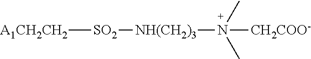 | |
| A₁—C₂H₄—SCH₂CH₂CO₂M | |
| A₁—C₂H₄—SCH₂CH₂N⁺(CH₃)₃•CH₃—SO₄⁻ | |
| A₂—SO₂N(C₂H₅)CH₂CO₂⁻•K⁺ | Formula (V) |
| A₂—SO₂—NH(CH₂)ₚ—N⁺(CH₃)₃•I⁻ | |
| A₂—SO₃⁻•NH₄⁺ | |
| A₂—SO₂N(C₂H₅)CH₂CO₂⁻•NH₄⁺ | |
| (A₃—C₂H₄O)₂—PO(ONH₄) | Formula (VI) |
| A₃—C₂H₄O—PO(OH)₂ | |

The composition of the present invention can be prepared by mixing an aqueous fluorosurfactant solution and an aqueous fluoro-free hydrotrope solution or mixing neat fluorosurfactant and neat fluoro-free hydrotrope. The resultant mixture is then diluted to the desired concentration with water. The weight ratio of the fluorosurfactant to the fluoro-free hydrotrope in the composition can be in the range of 1:10 to 10:1, or 1:8 to 8:1, or 1:5 to 5:1, or 1:3 to 3:1. The total concentration of the fluorosurfactant and the fluoro-free hydrotrope in the compositions is 0.0002-5 wt %, or 0.001-1 wt %. The concentration of the fluorosurfactant in the composition is 0.00005-2 wt %, or 0.0005-1 wt % to attain surface tensions of 15-35 mN/m. In some embodiments, the disclosed composition comprising a fluorosurfactant and a fluoro-free hydrotrope exhibits the same level of surface tension as a comparative consisting of fluorosurfactant, wherein the composition has only one third or one fourth the amount of fluorosurfactant compared to the comparative. In some embodiments, the disclosed composition comprising a fluorosurfactant and a fluoro-free hydrotrope, exhibits wetting and leveling characteristics comparable to a comparative consisting of fluorosurfactant, wherein the composition has only half the amount of fluorosurfactant compared to the comparative.

Another aspect of the present invention relates to a method of altering the surface behavior of an agent, comprising adding to the agent a composition comprising a fluorosurfactant and a fluoro-free hydrotrope, wherein the weight ratio of the fluorosurfactant to the fluoro-free hydrotrope is in the range of 1:10 to 10:1, or 1:8 to 8:1, or 1:5 to 5:1, or 1:3 to 3:1.

The fluoro-free hydrotrope comprises a compound selected from the group of compounds of formula (I), compounds of formula (II), and compounds of formula (III), described supra. The total concentration of the fluorosurfactant and the fluoro-free hydrotrope in the agent is 0.0002-5 wt %, or 0.001-1 wt %. The concentration of the fluorosurfactant in the agent is 0.00005-2 wt %, or 0.0005-1 wt %. Exemplary surface behavior of an agent that can be altered include, but is not limited to, wetting, penetration, spreading, leveling, flowing, emulsifying, dispersing, repelling, releasing, lubricating, etching, bonding, and stabilizing. Exemplary agents whose surface behavior can be altered by the addition of the composition disclosed hereinabove include, but are not limited to, coating compositions, lattices, polymers, floor finishes, inks, emulsifying agents, foaming agents, release agents, repellency agents, flow modifiers, film evaporation inhibitors, wetting agents, leveling agents, penetrating agents, cleaners, grinding agents, electroplating agents, corrosion inhibitors, etchant solutions, soldering agents, dispersion aids, antimicrobial agents, pulping aids, rinsing aids, polishing agents, personal care compositions, drying agents, antistatic agents, bonding agents, and mixtures thereof.

A further aspect of the present invention relates to a process comprising contacting an article with a composition comprising a fluorosurfactant and a fluoro-free hydrotrope, wherein the weight ratio of the fluorosurfactant to the fluoro-free hydrotrope is 1:10 to 10:1, or 1:8 to 8:1, or 1:5 to 5:1, or 1:3 to 3:1. The concentration of the fluorosurfactant in the composition is 0.00005-2 wt %, or 0.0005-1 wt %. The fluoro-free hydrotrope comprises a compound selected from the group of compounds of formula (I), compounds of formula (II), and compounds of formula (III).

In an embodiment, the composition used in the process comprising contacting an article, further comprises an agent disclosed hereinabove. The concentration of the fluorosurfactant in the agent is 0.00005-2 wt %, or 0.0005-1 wt %.

Suitable articles include: polymers, metals, wood, glass, ceramics, bricks, concretes, cements, natural or synthetic stones, tiles, paper, leather, and textile materials. Suitable polymers include: polycarbonates, polyesters (such as polyethylene terephthalate), polyolefins, polyurethanes, acrylics, polyamides (such as nylon 6, nylon 6,6, and nylon 6,12), polyimides, vinyl polymers (such as polyvinyl chloride), fluoropolymers, silicon polymers (such as polysilanes and polysiloxanes), amino resins, epoxy resins, and phenolic resins. The polymeric articles can be in the form of a fiber, a film, a sheet, a formed or molded part, a laminate, an extruded profile, a coated part, a foamed part, a bead, a particle, or a powder. Typical natural stones include granite and marble, and examples of synthetic stones include solid surface materials such as Corian® from DuPont and quartz surfaces such as Zodiaq® from DuPont.

The compositions of the present invention can be used in waxes, finishes, and polishes to improve wetting, leveling, and gloss for floors, furniture, shoes, and automotive care. The compositions of the present invention are useful in a variety of aqueous and non-aqueous cleaning products for glass, tile, marble, ceramics, linoleum, metal, stone, laminates, natural and synthetic rubbers, resins, plastics, fibers, and fabrics.

The compositions of the present invention can also be employed as additives in agricultural compositions containing herbicides, hormone growth regulators, parasiticides, insecticides, germicides, bactericides, nematocides, microbiocides, fungicides, miticides, defoliants, fertilizers, therapeutic agents, and antimicrobials, with one or more of the following functions: substrate wetting agent, adjuvant, foam inhibitor, dispersant, and emulsion stabilizer. The compositions of the present invention are also suitable as wetting agents for foliage, livestock dips, and livestock skins; as an ingredient in sanitizing, discoloring and cleaning compositions; and in insect repellent compositions.

The compositions of the present invention are suitable for the use in compositions for personal care products (such as shampoos, conditioners, creams, and rinses), cosmetic products for the skin (such as therapeutic or protective creams and lotions, oil and water repellent cosmetic powders, deodorants and anti-perspirants), nail polish, lipstick, toothpastes, fabric care products (such as stain pretreatments and/or stain removers for clothing, carpets and upholstery), laundry detergents, and rinse-aids (for car washes and in automatic dishwashers).

The compositions of the present invention are suitable for the use in the petroleum and gas industries as wetting agents and treatment agents to prevent and remove film evaporation and gas/oil blocking for gas, gasoline, jet fuel, solvents and hydrocarbons.

The compositions of the present invention are suitable for the use in printing inks, resist inks, developer solutions, photoresists, cleaning solutions, oxide etching compositions, and polishers in the manufacturing, processing, and handling of semiconductors and electronics.

The compositions of the present invention are useful as fire fighting agents, dry chemical fire extinguishers, and aerosol-type fire extinguishers.

The compositions of the present invention are suitable for the use as wetting agents, antifoaming agents, penetrating agents and emulsifying agents in textile and leather industries; lubricants for textiles, nonwoven fabrics and leather treatment; spreading and uniformity agents for fiber finishes; wetting agents for dyeing; binders in nonwoven fabrics; and penetration additives for bleaches.

The compositions of the present invention are further useful as thickening agents in mining industry, metal-working industry, pharmaceutical industry, household, cosmetic and personal products, photography and graphic arts.

The compositions of the present invention can be used as antifogging agents for glass surfaces and photography films, and as antistatic agents for magnetic tapes, phonograph records, floppy disks, disk drives, rubber compositions, PVC, polyester film, photography films, and as surface treatment agents for optical elements (such as glass, plastic, or ceramic beads).

The compositions of the present invention are also useful as foam control agents in polyurethane foams, spray-on oven cleaners, foamed kitchen and bathroom cleansers and disinfectants, aerosol shaving foams, and textile-treatment baths.

The compositions of the present invention are useful as emulsifying agents for polymerization, particularly of fluoromonomers, as latex stabilizers, as mold-release agents for silicones, photoemulsion stabilizers, inorganic particles, and pigments.

### EXAMPLES

### Materials and Methods

Fluoro-free hydrotropes benzyltriethylammonium chloride (BTAC), p-toluidinehydrochloride (PTHC), and sodium-p-toluenesulfonate (PTSNa) were obtained from Sigma-Aldrich; and 2,2′-azobis(N,N′-dimethyleneisobutyramidine)dihydrochloride (Vazo® 44) was obtained from E. I. du Pont de Nemours and Company, Wilmington, Del. Fluorosurfactant S1 is an anionic fluorosurfactant containing a mixture of a fluoroalkyl phosphate ammonium salt and a glycol ester, in which the fluoroalkyl chain comprises 2-16 carbon atoms, predominantly 8 carbon atoms. Fluorosurfactant S2 is a cationic fluorosurfactant containing 1-(3,3,4,4,5,5,6,6,7,7,8,8,9,9,10,10,10-heptadecafluorodecyl)-pyridinium, 4-methylbenzene sulfonate. Fluorosurfactant S3 is an anionic fluorosurfactant containing a mixture of a fluoroalkyl phosphate ammonium salt and a glycol ester, in which the fluoroalkyl chain comprises 6 carbon atoms. Fluorosurfactants S1, S2 and S3 were obtained from E. I. du Pont de Nemours and Company, Wilmington, Del. Fluorosurfactant S4 is a nonionic fluorosurfactant containing a mixture of fluoroalkyl ethoxylate in water, prepared as per the procedure described in U.S. Pat. No. 5,567,857.

### Preparation of Compositions Comprising a Fluorosurfactant and a Fluoro-Free Hydrotrope

A bulk solution of a fluoro-free hydrotrope described in Examples 1-7 was prepared by dissolving 0.04 g of the fluoro-free hydrotrope in 39.96 g of deionized water to obtain a 0.1 wt % solution. Similarly, a 0.1 wt % solution of a fluorosurfactant was prepared by dissolving 0.04 g of the fluorosurfactant in 39.96 g of water to obtain a 0.1 wt % solution. The mixtures were allowed to sonicate for 5 min.

Compositions (0.01 wt %) comprising a fluorosurfactant and a fluoro-free hydrotrope were prepared as described below:
a) For a 3:1 fluorosurfactant to fluoro-free hydrotrope ratio, 3.0 g of the 0.1 wt % fluorosurfactant and 1.0 g of the 0.1 wt % fluoro-free hydrotrope solution were added to a clean, tared centrifuge tube. The tube was filled with deionized water to bring the total weight of the solution up to 40.0 g to obtain a 0.01 wt % solution.
b) For a 1:1 fluorosurfactant to fluoro-free hydrotrope ratio, 2.0 g of the 0.1 wt % fluorosurfactant and 2.0 g of the 0.1 wt % fluoro-free hydrotrope solution were added to a clean, tared centrifuge tube. The tube was filled with deionized water to bring the total weight of the solution up to 40.0 g to obtain a 0.01 wt % solution.
c) For a 1:3 fluorosurfactant to fluoro-free hydrotrope ratio, 1.0 g of the 0.1 wt % fluorosurfactant and 3.0 g of the 0.1 wt % fluoro-free hydrotrope solution were added to a clean, tared centrifuge tube. The tube was filled with deionized water to bring the total weight of the solution up to 40.0 g to obtain a 0.01 wt % solution.
d) For a 0.01 wt % fluorosurfactant control, 4.0 g of the 0.1 wt % fluorosurfactant solution was added to a clean, tared centrifuge tube. The tube was filled with deionized water to bring the total weight of the solution to 40.0 g to obtain a 0.01 wt % solution.
e) For a 0.01 wt % fluoro-free hydrotrope control, 4.0 g of the 0.1 wt % fluoro-free hydrotrope solution was added to a clean, tared centrifuge tube. The tube was filled with deionized water to bring the total weight of the solution to 40.0 g to obtain a 0.01 wt % solution.

### Test Method 1. Surface Tension Measurements

The surface tensions of the fluorosurfactants were measured in fresh MILLIPORE® filtered water using the Wilhelmy plate method (Acosta, E. J. and Reinartz, S., U.S. Pat. No. 7,385,077) on an automated Krüss tensiometer (Model K11, Krüss USA, Nazareth, Pa.). MILLIPORE® filters are available from Millipore Corporation, Billerica, Mass.

A clean, dry 50 mL plastic beaker was filled with approximately 40 mL of the desired solution for the surface tension measurement. The beaker was placed on the sample platform of the Krüss K11 tensiometer.

The platinum surface tension probe was removed from the tensiometer hook and rinsed with deionized water and dried with the blue part of the flame from a propane torch. The probe was then air-cooled and reinserted onto the tensiometer hook. The surface tension measurements were performed for compositions comprising a fluorosurfactant and a fluoro-free hydrotrope of various ratios. It is preferred to start with the sample of deionized water, followed by the lowest to the highest fluorosurfactant to fluoro-free hydrotrope ratio.

### Test Method 2—Wetting and Leveling Test

The wetting and leveling ability of the samples was tested by adding each sample to a floor polish (RHOPLEX® 3829, Rohm & Haas, Spring House, Pa.) and applying the mixture to half of a 12 inch×12 inch (30.36 cm×30.36 cm) vinyl tile that had been stripped with a Comet® cleaner. A 1 wt % solution of the composition comprising a fluorosurfactant and a fluoro-free hydrotrope to be tested was prepared by dilution with deionized water. Following the manufacturer protocols, a 100 g portion of the RHOPLEX® 3829 formulation was prepared, followed by addition of 0.75 g of the 1 wt % composition solution comprising a fluorosurfactant and a fluoro-free hydrotrope, to provide a test floor polish.

The test floor polish was applied to a tile by placing a 3 mL portion of the test polish in the center of the tile, spreading the solution from top to bottom using an applicator, and finally placing a large “X” across half of the tile, using the applicator. The tile was allowed to dry for 30 min. A total of 5 coats was applied. After each coat, the tile was rated on a 1 to 5 scale (1 being the worst, 5 the best) on the surfactant's ability to promote wetting and leveling of the polish on the tile surface. The rating was determined based on comparison of a tile treated with the floor polish that contained no composition of this invention or leveling aids, according to the following scale:

Subjective Tile-Rating Scale
1 Uneven surface coverage of the film, significant streaking and surface defects
2 Visible streaking and surface defects, withdrawal of the film from the edges of the tile
3 Numerous surface defects and streaks are evident but, generally, film coats entire tile surface
4 Minor surface imperfections or streaking
5 No visible surface defects or streaks

### Example 1

This example describes the preparation and testing of compositions comprising anionic fluorosurfactant S1 and cationic fluoro-free hydrotrope p-toluidine hydrochloride (PTHC) with varying ratios.

By following the procedure described above, compositions comprising S1 and PTHC were prepared in ratios of 3:1, 1:1 and 1:3 to provide 0.01 wt % solutions with different amounts of fluorosurfactant. Surface tensions of the compositions and controls were measured according to Test Method 1 and the results are summarized in Table 2.

TABLE 2

| | | Fluorosurfactant | |
|---|---|---|---|
| | | present in the | Surface |
| | | composition | Tension |
| Sample | Composition | (wt %) | (mN/m) |
| Control | PTHC 100% | 0 | 62 |
| PTHC | | | |
| Control S1 | S1 100% | 0.0035 | 23.4 |
| 1.1 | S1:PTHC::3:1 | 0.0026 | 21.2 |
| 1.2 | S1:PTHC::1:1 | 0.0018 | 23.4 |
| 1.3 | S1:PTHC::1:3 | 0.0009 | 31.5 |

Table 2 shows that the samples 1.1, 1.2, and 1.3 comprising varying amounts of S1 and PTHC showed comparable reduction in the surface tension at a substantially reduced level of fluorosurfactant concentration, compared to the corresponding fluorosurfactant control S1.

### Example 2

This example describes the preparation and testing of compositions comprising anionic fluorosurfactant S1 and cationic fluoro-free hydrotrope benzyltriethylammonium chloride (BTAC) with varying ratios.

By following the procedure described above, compositions comprising S1 and BTAC were prepared in ratios of 3:1, 1:1 and 1:3 to provide 0.01 wt % solutions with different amounts of fluorosurfactant. Surface tensions of the compositions and controls were measured according to Test Method 1 and the results are summarized in Table 3.

TABLE 3

| | | Fluorosurfactant | |
|---|---|---|---|
| | | present in the | |
| | | composition | Surface tension |
| Sample | Composition | (wt %) | (mN/m) |
| Control BTAC | BTAC 100% | 0 | 71.7 |
| Control S1 | S1 100% | 0.0035 | 23.3 |
| 2.1 | S1:BTAC::3:1 | 0.0026 | 21.8 |
| 2.2 | S1:BTAC::1:1 | 0.0018 | 23.1 |
| 2.3 | S1:BTAC::1:3 | 0.0009 | 23.8 |

Table 3 shows that the samples 2.1, 2.2, and 2.3 comprising varying amounts of S1 and BTAC showed comparable reduction in the surface tension at a substantially reduced level of fluorosurfactant concentration, compared to the corresponding fluorosurfactant control S1.

### Example 3

This example describes the preparation and testing of compositions comprising anionic fluorosurfactant S1 and anionic fluoro-free hydrotrope sodium p-toluene sulfonate (PTSNa) with varying ratios.

By following the procedure described above, compositions comprising S1 and PTSNa were prepared in ratios of 3:1, 1:1 and 1:3 to provide 0.01 wt % solutions with different amounts of fluorosurfactant. Surface tensions of the compositions and controls were measured according to Test Method 1 and the results are summarized in Table 4.

TABLE 4

| | | Fluorosurfactant | |
|---|---|---|---|
| | | present in the | |
| | | composition | Surface tension |
| Sample | Composition | (wt %) | (mN/m) |
| Control PTSNa | PTSNa 100% | 0.01 | 73.2 |
| Control S1 | S1 100% | 0.0035 | 23.4 |
| 3.1 | S1:PTSNa::3:1 | 0.0026 | 21.5 |
| 3.2 | S1:PTSNa::1:1 | 0.0018 | 23.9 |
| 3.3 | S1:PTSNa::1:3 | 0.0009 | 27.9 |

Table 4 shows that the samples 3.1, 3.2, and 3.3 comprising varying amounts S1 and PTSNa showed comparable reduction in the surface tension at a substantially reduced level of fluorosurfactant concentration, compared to the corresponding fluorosurfactant control S1.

### Example 4

This example describes the preparation and testing of compositions comprising cationic fluorosurfactant S2 and anionic fluoro-free hydrotrope sodium p-toluene sulfonate (PTSNa) with varying ratios.

By following the procedure described above, compositions comprising S2 and PTSNa were prepared in ratios of 3:1, 1:1 and 1:3 to provide 0.01 wt % solutions with different amounts of fluorosurfactant. Surface tensions of the compositions and controls were measured according to Test Method 1 and the results are summarized in Table 5.

TABLE 5

| | | Fluorosurfactant | |
|---|---|---|---|
| | | present in the | |
| | | composition | Surface tension |
| Sample | Composition | (wt %) | (mN/m) |
| Control PTSNa | PTSNa 100% | 0 | 73.2 |
| Control S2 | S2 100% | 0.005 | 32.9 |
| 4.1 | S2:PTSNa::3:1 | 0.00375 | 22.4 |
| 4.2 | S2:PTSNa::1:1 | 0.0025 | 22.5 |
| 4.3 | S2:PTSNa::1:3 | 0.00125 | 31.5 |

Table 5 shows that the samples 4.1, 4.2, and 4.3 comprising varying amounts of S2 and PTSNa showed comparable reduction in the surface tension at a significantly reduced level of fluorosurfactant concentration, compared to the corresponding fluorosurfactant control S2.

### Example 5

This example describes the preparation and testing of compositions comprising anionic fluorosurfactant S3 and cationic fluoro-free hydrotrope p-toluidine hydrochloride (PTHC) with varying ratios.

By following the procedure described above, compositions comprising S3 and PTHC were prepared in ratios of 3:1, 1:1 and 1:3 to provide 0.01 wt % solutions with different amounts of fluorosurfactant. Surface tensions of the compositions and controls were measured according to Test Method 1 and the results are summarized in Table 6.

TABLE 6

| | | Fluorosurfactant | |
|---|---|---|---|
| | | present in the | |
| | | composition | Surface tension |
| Sample | Composition | (wt %) | (mN/m) |
| Control PTHC | PTHC 100% | 0 | 62.0 |
| Control S3 | S3 100% | 0.0035 | 23.4 |
| 5.1 | S3:PTHC::3:1 | 0.0026 | 19.0 |
| 5.2 | S3:PTHC::1:1 | 0.0018 | 20.0 |
| 5.3 | S3:PTHC::1:3 | 0.0009 | 22.8 |

Table 6 shows that the samples 5.1, 5.2, and 5.3 comprising varying amounts of S3 and PTHC showed comparable reduction in the surface tension at a significantly reduced level of fluorosurfactant concentration, compared to the corresponding fluorosurfactant control S3.

### Example 6

This example describes the preparation and testing of compositions comprising anionic fluorosurfactant S3 and cationic fluoro-free hydrotrope benzyltriethylammonium chloride (BTAC) with varying ratios.

By following the procedure described above, compositions comprising S3 and BTAC were prepared in ratios of 3:1, 1:1 and 1:3 to provide 0.01 wt % solutions with different amounts of fluorosurfactant. Surface tensions of the compositions and controls were measured according to Test Method 1 and the results are summarized in Table 7.

TABLE 7

| | | Fluorosurfactant | |
|---|---|---|---|
| | | present in the | |
| | | composition | Surface tension |
| Sample | Composition | (wt %) | (mN/m) |
| Control BTAC | BTAC 100% | 0 | 72.4 |
| Control S3 | S3 100% | 0.0035 | 26.3 |
| 6.1 | S3:BTAC::3:1 | 0.0026 | 16.5 |
| 6.2 | S3:BTAC::1:1 | 0.0018 | 16.8 |
| 6.3 | S3:BTAC::1:3 | 0.0009 | 18.7 |

Table 7 shows that the samples 6.1, 6.2, and 6.3 comprising varying amount of S3 and BTAC showed comparable reduction in the surface tension at a significantly reduced level of fluorosurfactant concentration, compared to the corresponding fluorosurfactant control S3.

### Example 7

This example describes the preparation and testing of compositions comprising nonionic fluorosurfactant S4 and cationic fluoro-free hydrotrope 2,2′-azobis(N,N′-dimethyeneisobutyramidine)dihydrochloride (Vazo® 44) with varying ratios.

By following the procedure described above, compositions comprising S4 and Vazo® 44 were prepared in ratios of 3:1, 1:1 and 1:3 to provide 0.01 wt % solutions with different amounts of fluorosurfactant. Surface tensions of the compositions and controls were measured according to Test Method 1 and the results are summarized in Table 8.

TABLE 8

| | | Fluorosurfactant | |
|---|---|---|---|
| | | present in the | Surface |
| | | composition | tension |
| Sample | Composition | (wt %) | (mN/m) |
| Control Vazo ® 44 | Vazo ® 44 100% | 0 | 72.5 |
| Control S4 | S4 100% | 0.005 | 19.9 |
| 7.1 | S4:Vazo ® 44::3:1 | 0.00375 | 20.2 |
| 7.2 | S4:Vazo ® 44::1:1 | 0.0025 | 20.4 |
| 7.3 | S4:Vazo ® 44::1:3 | 0.00125 | 21.6 |

Table 8 shows the samples 7.1, 7.2, and 7.3 comprising varying amounts of S4 and Vazo® 44 showed comparable reduction in the surface tension at a reduced level of fluorosurfactant concentration, compared to the corresponding fluorosurfactant control S4.

### Example 8

The compositions comprising anionic fluorosurfactant S1 and cationic fluoro-free hydrotrope benzyltriethylammonium chloride (BTAC) prepared as per Example 2 were evaluated for performance as wetting and leveling agents in a commercial floor polish according to Test Method 2. In a control, no leveling agent was added. A comparative experiment was performed using S1 as leveling agent.

All samples were measured at 75 ppm (microgram/g) loading and at the same time to nullify potential variations in room humidity and temperature. The results are listed in Table 9, where higher ratings indicate better performance.

TABLE 9
Ratings for Wetting and Leveling Test

| | | Coating No. | | | | | |
|---|---|---|---|---|---|---|---|
| | | 1 | 2 | 3 | 4 | 5 | |
| Sample | Composition | Rating | | | | | Average |
| | Control | 2 | 2 | 1 | 1 | 1 | 1.4 |
| Comparative | S1 | 2 | 3 | 4 | 4 | 4.5 | 3.5 |
| 8.1 | S1:BTAC::3:1 | 2 | 3 | 4 | 4.5 | 4.5 | 3.6 |
| 8.2 | S1:BTAC::1:1 | 2 | 3 | 3.5 | 3.5 | 3 | 3.0 |

Table 9 shows that the compositions comprising S1 and BTAC showed comparable wetting and leveling characteristics at a reduced level of fluorosurfactant concentration, compared to S1.

### Example 9

The compositions comprising anionic fluorosurfactant S3 and cationic fluoro-free hydrotrope benzyltriethylammonium chloride (BTAC) prepared as per Example 6 were evaluated for performance as wetting and leveling agents in a commercial floor polish according to Test Method 2. In a control, no leveling agent was added. A comparative experiment was performed using S3 as leveling agent.

All samples were measured at 75 ppm (microgram/g) loading and at the same time to nullify potential variations in room humidity and temperature. The results are listed in Table 10, where higher ratings indicate better performance.

TABLE 10
Ratings for Wetting and Leveling Test

| | | Coating No. | | | | | |
|---|---|---|---|---|---|---|---|
| | | 1 | 2 | 3 | 4 | 5 | |
| Sample | Composition | Rating | | | | | Average |
| | Control | 2 | 2 | 1 | 1 | 1 | 1.4 |
| Comparative | S3 | 2 | 3 | 3.5 | 5 | 4 | 3.5 |
| 9.1 | S3:BTAC::3:1 | 2 | 3 | 3.5 | 4.5 | 4 | 3.4 |
| 9.2 | S3:BTAC::1:1 | 2 | 3 | 4 | 4 | 3.5 | 3.3 |

Table 10 shows that the compositions comprising S3 and BTAC showed comparable wetting and leveling characteristics at a substantially reduced level of fluorosurfactant concentration, compared to S3.

What is claimed is:

1. A composition comprising a fluorosurfactant and a fluoro-free hydrotrope, wherein the weight ratio of the fluorosurfactant to the fluoro-free hydrotrope is in the range of 1:10 to 10:1.

2. The composition of claim 1, wherein the concentration of the fluorosurfactant is 0.00005-2 wt %.

3. The composition of claim 1, wherein the fluoro-free hydrotrope is selected from the group consisting of compounds of formula (I), compounds of formula (II), and compounds of formula (III), as shown below: 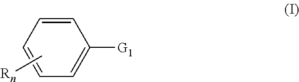
wherein:
R=R¹ or OR¹,
R¹=C₁-C₃ alkyl;
n=1, 2 or 3; and
G₁=-CO₂M, —SO₃M, —OSO₃M, —OPO₃(M₁)(M₂),
or —PO₃(M₁)(M₂),
M=H, Na, K, or NH₄,
M₁=H, Na, K, or NH₄, and
M₂=H, Na, K, or NH₄; 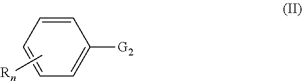
wherein:
R=R¹ or OR¹,
R¹=C₁-C₃ alkyl;
n=1, 2 or 3;
G₂=N⁺R²R³R⁴.X⁻
R²=H or C₁-C₃ alkyl,
R³=H or C₁-C₃ alkyl,
R⁴=H or C₁-C₃ alkyl, and
X=Cl, Br, or I; 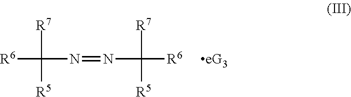
wherein:
R⁵=C₁-C₃ alkyl;
R⁶=C₁-C₃ alkyl;
R⁷=—(CH₂)ₐCO₂H, 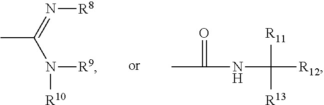
R⁸=H, —(CH₂)_{b}CO₂H, —(CH₂)_{c}OH, or C₁-C₃ alkylene,
R⁹=H, —(CH₂)_{b}CO₂H, —(CH₂)_{c}OH, or C₁-C₃ alkylene,
R¹⁰=H, —(CH₂)_{b}CO₂H, —(CH₂)_{c}OH, or C₁-C₃ alkylene,
a=1, 2, 3, or 4,
b=1, 2, 3, or 4,
c=1, 2, 3, or 4,
wherein R⁸ and R⁹, or R⁸ and R¹⁰, or R⁹ and R¹⁰ may be taken together with the nitrogen atom(s) to which they are attached to form a ring containing 5 to 7 atoms,
R¹¹=H or —(CH₂)_{d}OH,
R¹²=H or —(CH₂)_{d}OH,
R¹³=H or —(CH₂)_{d}OH,
d=1, 2, 3, or 4;
e=0, 1, 2, 3, or 4; and
G₃=HX, H₂SO₄, or H₂O,
X=Cl, Br, or I.

4. The composition of claim 1, wherein the fluorosurfactant is an anionic, a cationic, an amphoteric, or a nonionic fluorosurfactant.

5. A method of altering the surface behavior of an agent, comprising adding to the agent a composition comprising a fluorosurfactant and a fluoro-free hydrotrope, wherein the weight ratio of the fluorosurfactant to the fluoro-free hydrotrope is in the range of 1:10 to 10:1.

6. The composition of claim 5, wherein the concentration of the fluorosurfactant is 0.00005-2 wt %.

7. The method of claim 5, wherein the fluoro-free hydrotrope is selected from the group consisting of compounds of formula (I), compounds of formula (II), and compounds of formula (III), as shown below: 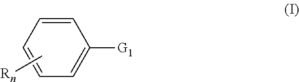
wherein:
R=R¹ or OR¹,
R¹=C₁-C₃ alkyl;
n=1, 2, or 3; and
G₁=-CO₂M, —SO₃M, —OSO₃M, —OPO₃(M₁)(M₂),
or —PO₃(M₁)(M₂),
M=H, Na, K, or NH₄,
M₁=H, Na, K, or NH₄, and
M₂=H, Na, K, or NH₄; 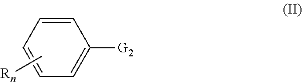
wherein:
R=R¹ or OR¹,
R¹=C₁-C₃ alkyl;
n=1, 2 or 3;
G₂=N⁺R²R³R⁴.X⁻
R²=H or C₁-C₃ alkyl;
R³=H or C₁-C₃ alkyl;
R⁴=H or C₁-C₃ alkyl; and
X=Cl, Br, or I; 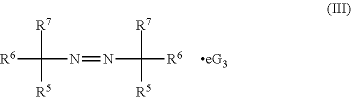
wherein:
R⁵=C₁-C₃ alkyl;
R⁶=C₁-C₃ alkyl;
R⁷=—(CH₂)ₐCO₂H, 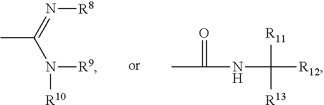
R⁸=H, —(CH₂)_{b}CO₂H, —(CH₂)_{c}OH, or C₁-C₃ alkylene,
R⁹=H, —(CH₂)_{b}CO₂H, —(CH₂)_{c}OH, or C₁-C₃ alkylene,
R¹⁰=H, —(CH₂)_{b}CO₂H, —(CH₂)_{c}OH, or C₁-C₃ alkylene,
a=1, 2, 3, or 4,
b=1, 2, 3, or 4,
c=1, 2, 3, or 4,
wherein R⁸ and R⁹, or R⁸ and R¹⁰, or R⁹ and R¹⁰ may be taken together with the nitrogen atom(s) to which they are attached to form a ring containing 5 to 7 atoms,
R¹¹=H or —(CH₂)_{d}OH,
R¹²=H or —(CH₂)_{d}OH, and
R¹³=H or —(CH₂)_{d}OH,
d=1, 2, 3, or 4;
e=0, 1, 2, 3, or 4; and
G₃=HX, H₂SO₄, or H₂O,
X=Cl, Br, or I.

8. The method of claim 5, wherein the fluorosurfactant is an anionic, a cationic, an amphoteric, or a nonionic fluorosurfactant.

9. The method of claim 5, wherein the agent is selected from the group consisting of coating compositions, lattices, polymers, floor finishes, inks, emulsifying agents, foaming agents, release agents, repellency agents, flow modifiers, film evaporation inhibitors, wetting agents, leveling agents, penetrating agents, cleaners, grinding agents, electroplating agents, corrosion inhibitors, etchant solutions, soldering agents, dispersion aids, antimicrobial agents, pulping aids, rinsing aids, polishing agents, personal care compositions, drying agents, antistatic agents, bonding agents, and mixtures thereof.

10. A process comprising contacting an article with a composition comprising a fluorosurfactant and a fluoro-free hydrotrope, wherein the weight ratio of the fluorosurfactant to the fluoro-free hydrotrope is in the range of 1:10 to 10:1.

11. The composition of claim 10, wherein the concentration of the fluorosurfactant is 0.00005-2 wt %.

12. The process of claim 10, wherein the fluoro-free hydrotrope comprises a compound selected from the group of compounds of formula (I), compounds of formula (II), and compounds of formula (III): 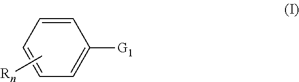
wherein:
R=R¹ or OR¹,
R¹=C₁-C₃ alkyl;
n=1, 2 or 3; and
G₁=-CO₂M, —SO₃M, —OSO₃M, —OPO₃(M₁)(M₂),
or —PO₃(M₁)(M₂),
M=H, Na, K, or NH₄,
M₁=H, Na, K, or NH₄, and
M₂=H, Na, K, or NH₄; 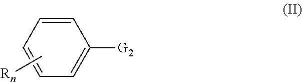
wherein:
R=R¹ or OR¹,
R¹=C₁-C₃ alkyl;
n=1, 2 or 3;
G₂=N⁺R²R³R⁴.X⁻
R²=H or C₁-C₃ alkyl;
R³=H or C₁-C₃ alkyl;
R⁴=H or C₁-C₃ alkyl; and
X=Cl, Br, or I; 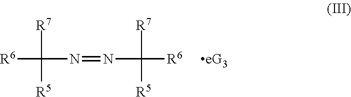
wherein:
R⁵=C₁-C₃ alkyl;
R⁶=C₁-C₃ alkyl;
R⁷=—(CH₂)ₐCO₂H, 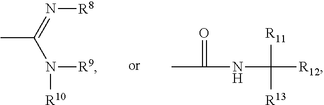
R⁸=H, —(CH₂)_{b}CO₂H, —(CH₂)_{c}OH, or C₁-C₃ alkylene,
R⁹=H, —(CH₂)_{b}CO₂H, —(CH₂)_{c}OH, or C₁-C₃ alkylene,
R¹⁰=H, —(CH₂)_{b}CO₂H, —(CH₂)_{c}OH, or C₁-C₃ alkylene,
a=1, 2, 3, or 4,
b=1, 2, 3, or 4,
c=1, 2, 3, or 4,
wherein R⁸ and R⁹, or R⁸ and R¹⁰, or R⁹ and R¹⁰ may be taken together with the nitrogen atom(s) to which they are attached to form a ring containing 5 to 7 atoms,
R¹¹=H or —(CH₂)_{d}OH,
R¹²=H or —(CH₂)_{d}OH, and
R¹³=H or —(CH₂)_{d}OH,
d=1, 2, 3, or 4;
e=0, 1, 2, 3, or 4; and
G₃=HX, H₂SO₄, or H₂O,
X=Cl, Br, or I.

13. The process of claim 10, wherein the fluorosurfactant is an anionic, a cationic, an amphoteric, or a nonionic fluorosurtactant.

14. The process of claim 10, wherein the composition further comprises an agent.

15. The process of claim 14, wherein the agent is selected from the group consisting of coating compositions, lattices, polymers, floor finishes, inks, emulsifying agents, foaming agents, release agents, repellency agents, flow modifiers, film evaporation inhibitors, wetting agents, leveling agents, penetrating agents, cleaners, grinding agents, electroplating agents, corrosion inhibitors, etchant solutions, soldering agents, dispersion aids, antimicrobial agents, pulping aids, rinsing aids, polishing agents, personal care compositions, drying agents, antistatic agents, bonding agents, and mixtures thereof.

16. The process of claim 10, wherein the article comprises a material selected from the group consisting of polymers, metals, wood, glass, ceramics, bricks, concretes, cements, natural or synthetic stones, tiles, paper, leather, and textile materials.

17. The process of claim 16, wherein the article is in the form of a fiber, a film, a sheet, a formed or molded part, a laminate, an extruded profile, a coated part, a foamed part, a bead, a particle, or a powder and the material is a polymer.

18. An article produced by the process of claim 10.
